(19) United States
(12) Patent Application Publication
(10) Pub. No.: US 2023/0119803 A1
(43) Pub. Date: Apr. 20, 2023

(54) COSMETIC COMPOSITION OBTAINED BY MIXING TWO SILANE BLENDS

(71) Applicant: Henkel AG & Co. KGaA, Duesseldorf (DE)

(72) Inventors: Phillip JAISER, Langenfeld (DE); Torsten LECHNER, Langenfeld (DE); Juergen SCHOEPGENS, Schwalmtal (DE); Marc NOWOTTNY, Moenchengladbach (DE); Carsten MATHIASZYK, Essen (DE); Avni TAIRI, Duisburg (DE); Carolin KRUPPA, Hilden (DE); Andreas WALTER, Ratingen (DE)

(73) Assignee: Henkel AG & Co. KGaA, Duesseldorf (DE)

(21) Appl. No.: 17/961,409

(22) Filed: Oct. 6, 2022

(30) Foreign Application Priority Data
Oct. 7, 2021 (DE) 102021211314.1

(51) Int. Cl. A61K 8/898; A61Q 5/06

(52) U.S. Cl. CPC A61K 8/898; A61Q 5/065; A61K 2800/805

(57) ABSTRACT

A process for preparing a cosmetic composition useful for treating keratinous material, in particular human hair, is disclosed. The process comprises preparing a first agent (A) by reacting one or more aminoalkyl-C₁-C₆ alkoxysilanes (a1) with water (a2), preparing a second agent (B) by reacting one or more alkyl-C₁-C₆ alkoxysilanes (b1) with water (b2), and mixing together the first agent (A) and the second agent (B), thereby preparing the cosmetic composition. A cosmetic composition prepared by the process, as well as a method of using the cosmetic composition for treating keratinous material (e.g. coloring human hair) are also disclosed.

### CROSS-REFERENCE TO RELATED APPLICATION

This application claims priority to German Patent Application No. 102021211314.1, filed Oct. 7, 2021, which is incorporated herein by reference in its entirety.

### TECHNICAL FIELD

The present application relates generally to cosmetic compositions for treating keratinous material and, more specifically, to a process for preparing cosmetic compositions using two agents each comprising a different silane blend, cosmetic compositions prepared via the process, and methods of using the cosmetic compositions for treating keratinous material (e.g. coloring human hair).

### BACKGROUND

The change in shape and color of keratin fibers, especially hair, is an important area of modem cosmetics. To change the hair color, the expert knows various colouring systems depending on colouring requirements. Oxidation dyes are usually used for permanent, intensive dyeing with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing obtained with direct dyes have a shorter shelf life and quicker washability. Dyes with direct colorings usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents comprising surfactants. Various products of this type are available on the market under the name hair mascara.

EP 2168633 B1 deals with the task of producing long-lasting hair colorations using pigments. The document illustrates that when a combination of pigment, organic silicon compound, hydrophobic polymer and a solvent is used on hair, it is possible to produce colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these hydrolysis or condensation products are applied to keratinous material, a film or coating is formed on the keratinous material, which completely envelops the keratinous material and in this way strongly influences the properties of the keratinous material. Possible areas of application include permanent styling or permanent shape modification of keratin fibers. In this process, the keratin fibers are mechanically shaped into the desired form and then fixed in this form by forming the coating described above. Another particularly suitable application is the coloring of keratin material; in this application, the coating or film is produced in the presence of a coloring compound, for example a pigment. The film colored by the pigment remains on the keratin material or keratin fibers, and surprisingly wash-resistant colorations result.

The great advantage of the alkoxy silane-based dyeing principle is that the high reactivity of this class of compounds enables very fast coating. This means that extremely good coloring results can be achieved after very short application periods of just a few minutes. In addition to these advantages, however, the high reactivity of alkoxy silanes also has some disadvantages. Thus, even minor changes in production and application conditions, such as changes in humidity and/or temperature, can lead to sharp fluctuations in product performance. Most importantly, the work leading to this present disclosure has shown that the alkoxy silanes are extremely sensitive to the conditions encountered in the manufacture of the keratin treatment agents.

Further analytical studies have shown that complex hydrolysis and condensation reactions take place during the preparation of various silane or siloxane mixtures and blends, leading to oligomeric products of different molecular size and degree of crosslinking, depending on the reaction conditions selected. In this context, it has been found that the molecular weight of these silane oligomers can have a major influence on the subsequent product properties. If wrong conditions are selected during production, this can lead to the formation of silane condensates that are too large or too small, which negatively affects the subsequent product performance, especially the subsequent dyeing capacity on the keratin material.

### BRIEF SUMMARY

A process for preparing a cosmetic composition is provided. The cosmetic composition is useful for treating keratinous material (e.g. coloring human hair). The process comprises preparing a first agent (A) by reacting one or more aminoalkyl-C₁-C₆ alkoxysilanes (a1) with water (a2), preparing a second agent (B) by reacting one or more alkyl-C₁-C₆ alkoxysilanes (1)1) with water (b2), and mixing together the first agent (A) and the second agent (B). In preparing the first agent (A), each of the one or more aminoalkyl-C₁-C₆ alkoxysilanes (a1) is independently selected from (3-aminopropyl)triethoxysilane, (3-aminopropyl)trimethoxysilane, (2-aminoethyl)triethoxysilane, (2-aminoethyl)trimethoxysilane, (3-dimethylaminopropyl)triethoxysilane, (3-dimethylaminopropyl)trimethoxysilane, (2-dimethylaminoethyl)triethoxysilane, and (2-dimethylaminoethyl)trimethoxysilane. In preparing the second agent (B), each of the one or more alkyl-C₁-C₆ alkoxysilanes (b1) is independently selected from a Group (b1): methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, vinyltrimethoxysilane, vinyltriethoxysilane tetramethoxysilane, and tetraethoxysilane. The first agent (A) is substantially free from alkyl-C₁-C₆ alkoxysilanes, and the second agent (B) is substantially free from aminoalkyl-C₁-C₆ alkoxysilanes.

Also provided is a cosmetic composition for treating keratinous material. The cosmetic composition is prepared according to the process.

A method of treating keratinous material is further provided. The method comprises applying the cosmetic composition to the keratinous material. In some embodiments, the method is further defined as a method of coloring human hair.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present application provides a process for preparing a cosmetic composition obtained by mixing the two agents (A) and (B). The two agents (A) and (B) are two different silane blends. The first agent (A) is obtained by reacting one or more reactive silanes of the aminoalkyl-C₁-C₆ alkoxysilane type (a1) with water (a2). The second agent (B) is obtained by reacting one or more reactive silanes of the alkyl-C₁-C₆ alkoxysilane type (b1) with water (b2). A characteristic feature of both agents is that agent (A) does not contain any organic C₁-C₆ alkoxysilanes from group (b1) and agent (B) does not contain any organic C₁-C₆ alkoxysilanes from group (a1).

A second object of the present disclosure is a cosmetic composition obtained via the manufacturing process of the first object of the disclosure.

A third object is the use of a cosmetic composition obtained via the manufacturing process of the first object of the disclosure for treating keratinous material, in particular for coloring keratinous material, in particular for coloring human hair.

It was the task of the present application to find optimized agents and methods for the treatment of human hair. The mixtures of alkoxy siloxanes used in these agents or processes should be prepared in a targeted manner so that the optimum application properties can be achieved in a subsequent application. Agents prepared in this way should have an ideal degree of crosslinking or siloxane oligomers with optimal molecular weight distribution, resulting in improved dyeing performance. In this context, it was particularly important to make the production of the agents reproducible. In this way, the agents, when applied in a dyeing process, should result in dyeing with higher color intensity and improved fastness properties while standardizing the color result. In particular, wash fastness and rub fastness should be improved in a reproducible way. Furthermore, agents prepared in this way should be particularly stable in storage, and the subsequent colorimetric potential of the agents should not depend on their storage time.

Surprisingly, it has now been found that the aforementioned task can be excellently solved if cosmetic compositions are applied to the keratin material, which are prepared by mixing two agents (A) and (B) before application. The two agents (A) and (B) represent different silane blends, in each of which C₁-C₆ alkoxy silanes of a particular group are separately hydrolyzed and oligomerized. Specific amounts of water can be used in both agents (A) and (B) for the two hydrolysis reactions.

A first object of the present disclosure is a process for the preparation of a cosmetic composition for the treatment of keratinous material, in particular human hair, obtained by mixing a first agent (A) with a second agent (B), wherein
the first agent (A) is obtained by reacting one or more organic C₁-C₆ alkoxysilanes a1) with water (a2),
wherein the organic C₁-C₆ alkoxysilane or alkoxysilanes (a1) are selected from the group of (3-aminopropyl)triethoxysilane, (3-aminopropyl)trimethoxysilane, (2-aminoethyl)triethoxysilane, (2-aminoethyl)trimethoxysilane, (3-dimethylaminopropyl)triethoxysilane, (3-dimethylamino-propyl)tri-methoxysilane, (2-dimethylaminoethyl)triethoxysilane and (2-dimethylaminoethyl)trimethoxysilane, and
   the second agent (B) is obtained by reacting one or more organic C₁-C₆ alkoxysilanes (b1) with water (b2),
wherein the organic C₁-C₆ alkoxysilane(s) (b1) is (are) selected from the group of methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, vinyltrimethoxysilane, vinyltriethoxysilane tetramethoxysilane and tetraethoxysilane,
wherein
   the agent (A) does not contain any organic C₁-C₆-alkoxysilanes from group (b1) and
   the agent (B) does not contain any organic C₁-C₆-alkoxysilanes from group (a1).

In abbreviated form, the first subject matter is a process for preparing a cosmetic composition obtained by mixing a first agent (A) with a second agent (B), wherein
the first agent (A) is obtained by reacting one or more organic C₁-C₆ alkoxysilanes (a1) with water (a2),
wherein the organic C₁-C₆ alkoxysilane or alkoxysilanes (a1) are selected from the group of (3-aminopropyl)triethoxysilane, (3-aminopropyl)trimethoxysilane, (2-aminoethyl)triethoxysilane, (2-aminoethyl)trimethoxysilane, (3-dimethylaminopropyl)triethoxysilane, (3-dimethylamino-propyl)tri-methoxysilane, (2-dimethylaminoethyl)triethoxysilane and (2-dimethylaminoethyl)trimethoxysilane, and
   the second agent (B) is obtained by reacting one or more organic C₁-C₆ alkoxysilanes (b1) with water (b2),
wherein the organic C₁-C₆ alkoxysilane(s) (b1) is (are) selected from the group of methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, vinyltrimethoxysilane, vinyltriethoxysilane tetramethoxysilane and tetraethoxysilane,
wherein
   the agent (A) does not contain any organic C₁-C₆-alkoxysilanes from group (b1) and
   the agent (B) does not contain any organic C₁-C₆-alkoxysilanes from group (a1).

The first agent (A) is obtained by reacting one or more organic C₁-C₆ alkoxysilanes (a1) with water (a2), said C₁-C₆ alkoxysilanes (a1) being of the aminoalkyl-C₁-C₆-alkoxysilane type.

The second agent (B) is obtained by reacting one or more organic C₁-C₆ alkoxysilanes (b1) with water (b2), said C₁-C₆ alkoxysilanes (b1) being of the alkyl-C₁-C₆ alkoxysilane type.

Surprisingly, it was found that by hydrolyzing and condensing the two silane types separately in both agents (A) and (B), it becomes possible to control the reactions of the two differently reactive silane types much better. After mixing the two agents (A) and (B), it was possible in this way to obtain a cosmetic composition which, when applied to the hair, achieves particularly good application properties. Surprisingly, this cosmetic composition had particularly good storage stability compared with compositions prepared by other methods, and that the results obtained with this composition, especially color results, could be reproduced quite well.

When subsequently applied to the hair, cosmetic compositions prepared in this way form particularly stable and reproducible films or coatings whose properties are independent of the duration of application of the composition. Furthermore, it has been shown that the cosmetic compositions prepared by mixing the two agents (A) and (B), when used in a dyeing process, resulted in very intense and uniform dyeing with very good hiding power, rub fastness and wash fastness.

### Keratinic Material

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair in particular.

### Cosmetic Composition for the Treatment of Keratinous Material

A cosmetic composition for treating keratinous material is understood to mean, for example, an agent for coloring the keratinous material, an agent for reshaping or shaping keratinous material, in particular keratinous fibers, or also an agent for conditioning or caring for the keratinous material. The cosmetic compositions prepared via the process as contemplated herein show particularly good suitability for coloring keratinous material, in particular for coloring keratinous fibers, which are especially preferably human hair.

The term “cosmetic composition for coloring” is used in the context of the present disclosure for a coloring of the keratin material, in particular of the hair, caused by the use of coloring compounds such as pigments, mica, thermochromic and photochromic dyes, direct dyes and/or oxidation dyes. In this staining process, the aforementioned colorant compounds are deposited in a particularly homogeneous and smooth film on the surface of the keratin material or diffuse into the keratin fiber. The film is formed in situ by oligomerization or condensation of the organic silicon compound(s), with the colorant compound(s) interacting with and being incorporated into or surrounded by this film or coating.

### Cosmetic Composition

The cosmetic composition of the first article of the present disclosure is obtained by mixing the two agents (A) and (B). For mixing, the two agents (A) and (B) can, for example, be stirred together, shaken together or mixed together in some other way. For example, for the purpose of mixing, agent (A) may be presented and agent (B) added, or agent (B) may be presented and subsequently agent (A) added.

The cosmetic composition resulting from the mixing of the two agents (A) and (B) can be stored for a certain period of days, weeks or months before being applied to the keratin material. This may be the case, for example, when the cosmetic composition is packaged and provided to the user or hairdresser as part of a cosmetic product or multi-component packaging unit for the treatment of the keratin material.

It is also possible to apply the cosmetic composition resulting from mixing the two agents (A) and (B) to the keratinous material promptly after mixing.

Furthermore, it is also possible to additionally mix the cosmetic composition resulting from the mixing of the two agents (A) and (B) with one or more further cosmetic compositions prior to application to the keratinous material.

Finally, it is also possible and as contemplated herein if the cosmetic composition obtained after mixing agents (A) and (B) undergoes a further reaction in which, for example, continued hydrolysis, oligomerization and/or condensation may take place, carried out with or using a catalyst. This embodiment is particularly preferred.

### Agent (A)

The first agent (A) is obtained by reacting one or more organic C₁-C₆ alkoxysilanes (a1) with water (a2).
Organic C₁-C₆ Alkoxy Silanes (a1) in the Agent (A)

The organic C₁-C₆-alkoxy-silanes (a1) are organic, non-polymeric silicon compounds selected from aminoalkyl-C₁-C₆ alkoxysilanes. Specifically, the organic C₁-C₆-alkoxy-silanes (a1), which may be referred to as the aminoalkyl-C₁-C₆ alkoxysilanes (a1), are typically selected from the group of (3-aminopropyl) triethoxysilane, (3-amino-propyl) trimethoxysilane, (2-aminoethyl) triethoxysilane, (2-aminoethyl) trimethoxysilane, (3-dimethyl-aminopropyl) triethoxysilane, (3-dimethylaminopropyl) trimethoxysilane, (2-dimethylamino-ethyl) triethoxysilane, and (2-dimethylaminoethyl) trimethoxysilane.

The organic C₁-C₆ alkoxy silanes (a1) which are particularly suitable for solving the problem as contemplated herein are
(3-Aminopropyl)triethoxysilane:

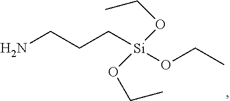

(3-Aminopropyl)trimethoxysilane:

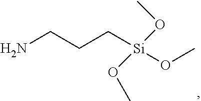

(2-Aminoethyl)triethoxysilane:

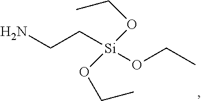

(2-Aminoethyl)trimethoxysilane:

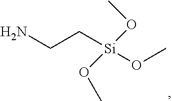

(3-Dimethylaminopropyl)triethoxysilane:

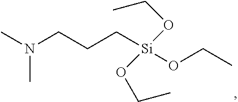 [SMILES: CCO[Si](CCCN(C)C)(OCC)OCC]

(3-Dimethylaminopropyl)trimethoxysilane:

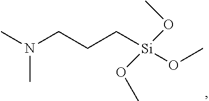

(2-Dimethylaminoethyl)triethoxysilane:

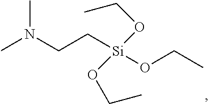

and/or
(2-Dimethylaminoethyl)trimethoxysilane:

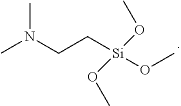

The aforementioned organic silicon compounds (a1) are commercially available. (3-aminopropyl)trimethoxysilane, for example, can be purchased from Sigma-Aldrich. Also (3-aminopropyl)triethoxysilane is commercially available from Sigma-Aldrich.

In the preparation of the first agent (A) by one or more organic C₁-C₆ alkoxysilanes (a1) are reacted with water (a2). Particularly good results were obtained when (a1) (3-aminopropyl) triethoxysilane was used as the C₁-C₆ organic alkoxysilane. Most preferably, therefore, the first agent (A) is obtained by reacting 3-aminopropyl)tri-ethoxysilane (a1) with water (a2).

Selective Hydrolysis of the Organic C₁-C₆ Alkoxysilanes (a1) in the Agent (A) by Addition of Water (a2)

In the preparation of the agent (A), one or more organic C₁-C₆ alkoxysilanes (a1) are mixed with water (a2) to initiate selective hydrolysis and, as a result, pre-condensation.

Since only aminoalkyl-C₁-C₆-alkoxysilanes oligomerize with each other in agent (A), an oligomer is formed in agent (A) which is formed exclusively by selective hydrolysis and condensation of the aminoalkyl-C₁-C₆-alkoxysilanes (a1). Thus, all monomer components of the oligomer or reaction product present in the agent (A) contain an aminoalkyl group.

The reaction with the water (a2) can be initiated, for example, by dropping or adding the water to the organic C₁-C₆ alkoxysilane(s) (a1). Optionally, a solvent may be present during the reaction or hydrolysis.

Also as contemplated herein, the hydrolysis is started by adding the amount of water (a2) and the aminoalkyl-C₁-C₆-alkoxysilanes (a1).

Mixing with water (a2) can be done at room temperature. For the application properties of the subsequent cosmetic agent, it can be of further advantage if the mixture of organic C₁-C₆ alkoxysilanes (a1) and optionally solvent is heated to a temperature of from about 30 to about 80° C., preferably from about 40 to about 75° C., more preferably from about 45 to about 70° C. and very particularly preferably from about 50 to about 65° C., before the water (a2) is added.

Adjustment of the preferred and particularly preferred temperature ranges can be accomplished by tempering the reaction vessel or reactor. For example, the reaction vessel or reactor may be surrounded from the outside by a temperature control bath, which may be a water bath or silicone oil bath, for example.

If the reaction is carried out in a double-walled reactor, a temperature-controlled liquid can also be passed through the space formed by the two walls surrounding the reaction chamber.

Since the hydrolysis reaction is exothermic, it has been found to be particularly advantageous to stir or mix the reaction mixture for improved heat dissipation. It is therefore particularly preferred that the water be added while stirring. The reaction, which is now initiated by the addition of water, continues to proceed exothermically, so that the reaction mixture remains at the preferred temperature ranges indicated above or may even heat up further without any further addition of energy. It is preferred if the additional heating due to the exothermic nature of the reaction remains within a range of from about 5 to about 20° C. If the reaction mixture heats up beyond this range, it is advantageous to cool the mixture.

The water can be added continuously, in partial quantities or directly as a total quantity. To ensure adequate temperature control, the amount and rate of water added is preferentially adjusted. Depending on the amount of silanes used, the addition and reaction can take place over a period of from about 2 minutes to about 72 hours.

The addition of the water (a2) initiates a selective hydrolysis of the organic C₁-C₆ alkoxysilanes (a1). For the purposes of the present disclosure, targeted hydrolysis means hydrolyzing some, but not all, of the C₁-C₆ alkoxy groups present in the C₁-C₆ organic alkoxysilanes.

The alkoxysilane-water ratio has an influence on the crosslinking within the siloxane network. The degree of crosslinking can be described by so-called T-structures. Here stands T₀ for an unreacted monomer, e.g. 3-aminopropyltriethoxysilane. In the case of a T₁-bond, a siloxane bond exists between two alkoxysilanes, and the two siloxanes are not bonded to any other alkoxysilane. In an T₂-bond, one alkoxysilane is bonded to exactly two others. A T₃-bond describes a siloxane that is bonded to three other siloxanes.

By using different mole ratios of alkoxysilanes (a1) to water (a2), the distribution of these different T structures can be influenced. A high ratio, i.e. less water, leads to less cross-linked structures, whereas a low ratio, i.e. a larger amount of water, leads to more crosslinking.

The quantitative determination of the T0 structures, T1 structures, T2 structures and T3 structures included in the agent in each case can be carried out, for example, by employing quantitative ²⁹Si NMR spectroscopy.
Use of NMR sample tubes
Device: Agilent, 600 MHz
Standard: TMS (tetramethylsilane)
Relaxation accelerator: Chromium(III) acetylacetonate
   ²⁹Si-NMR spectra were recorded in chloroform from each of the compositions. Measurements were taken on the day of production and after a defined period of time. By using the relaxation accelerator, the integrals of the individual signals became comparable with each other. The sum over all integrals was set equal to 100 mol %. For the quantitative determination, the area of each individual signal was related to the total sum over all integrals.

For example, the spectra can be measured using the procedure described in Journal of Organometallic Chemistry 625 (2001), 208-216.

The amount of water used to prepare the agent (A) is preferably equal to the molar amount of water determined by equation (G-1)

X=[(n_{I}(alkoxysilanes (a1))×n_{II}(alkoxy groups)]/n(H₂O(a2)) (G-1),

where
n(H₂O) is the molar amount of water (a2) used in the agent (A),
n_{I} is the molar amount of the organic C₁-C₆-alkoxysilanes (a1) used in the agent (A),
n_{II} is the number of C₁-C₆ alkoxy groups per organic C₁-C₆ alkoxysilane (a1) used in agent (A), and
X represents a number from 0.1 to 100.0.

In other words, the index number X indicates the molar ratio of the total number of moles of hydrolyzable C₁-C₆ alkoxy groups (a1) in the agent (A), which is related to the molar amount of water (a2) used in the agent (A).

nI indicates the total molar amount of C₁-C₆ organic alkoxysilanes (a1) used in the agent (A). If only one C₁-C₆ alkoxysilane of a particular structure is used, the total molar amount n_{I} is equal to the molar amount of the C₁-C₆ alkoxysilane used.

However, if a mixture of C₁-C₆ alkoxysilanes (a1) is used to prepare the agent (A), the total molar amount is the sum of the individual molar amounts of each C₁-C₆ alkoxysilane used.

Furthermore, n_{II} indicates the number of C₁-C₆ alkoxy groups per C₁-C₆ organic alkoxysilane (a1) used in the agent (A). If only one C₁-C₆ alkoxysilane of a given structure is used, n_{II} corresponds to the number of C₁-C₆ alkoxy groups present in that molecule.

However, if a mixture of C₁-C₆ alkoxysilanes (a1) is used to prepare the agent (A), the number of C₁-C₆ alkoxy groups (a1) of each C₁-C₆ alkoxysilane enters the equation.

If several C₁-C₆ alkoxysilanes (a1) are used to prepare the agent (A), the above equation expands to equation (G-1′) by forming the respective summands:

$$X = \frac{\sum [ nI ( Alkoxysilan ( a 1 ) ) \times nII ( Alkoxygruppen ) ]}{n ( H 2 O ) ( a 2 )} \qquad ( G - {1}^{′} )$$

### Sample Calculation

In the preparation of agent (A), 15.0 g of 3-aminopropyltriethoxysilane (C₉H₂₃NO₃Si=221.37 g/mol) and 15.0 g of 3-aminopropyltrimethoxysilane (C₆H17NO3Si=179.29 g/mol) were mixed together. Then, 6.06 g of water (18.015 g/mol) was added with stirring.
15.0 g 3-aminopropyltriethoxsilane (AMEO)=0.068 mol
3-Aminopropyltriethoxsilane has 3 hydrolyzable alkoxy groups per molecule.
15.0 g 3-aminopropyltrimethoxysilane (AMMO)=0.085 mol
3-Aminopropyltrimethoxysilane has 3 hydrolyzable alkoxy groups per molecule.
3.0 g water=0.167 mol

When two different C₁-C₆ alkoxysilanes are used, the value X is calculated by applying the formula (G-1′) under formation of appropriate sums, i.e.

$$X = \frac{[ n ( AMEO ) \times 3 ] + ( n ( AMMO ) \times 3 ]}{n ( H 2 O ) ( a 2 )} \\ X = \frac{[ ( 0.068 ) \times 3 ] + [ ( 0.085 ) \times 3 ]}{0.167} = 2.75$$

If further or other mixtures of C₁-C₆ alkoxy silanes (a1) are used, the formula (G-1) or (G-1′) is adapted accordingly or extended by the corresponding summands.

The degree of crosslinking set during the preparation of the agent (A) by adding the appropriate amount of water (a2) influences the application properties of the coating produced during subsequent application to the keratin material. A particularly stable, and resistant film could be produced when first agent (A) is obtained by reacting with an amount of water equal to the molar amount of water determined by equation (G-1), where X is a number from about 0.1 to about 100.0, preferably from about 1.0 to about 50.0, more preferably from about 1.2 to about 30.0, still more preferably from about 1.3 to about 10, and most preferably from about 1.8 to about 4.5.

In the context of a further particularly preferred embodiment, a process as contemplated herein is therefore wherein the organic C₁-C₆-alkoxysilanes (a1) are reacted in the agent (A) with an amount of water (a2) corresponding to the molar amount of water determined according to equation (G-1):

X=[(n_{I}(alkoxysilanes(a1))×n_{II}(alkoxy groups)]/n(H₂O(a2)) (G-1),

where
n(H₂O) is the molar amount of water (a2) used in the agent (A),
n_{I} is the molar amount of the organic C₁-C₆-alkoxysilanes (a1) used in the agent (A),
n_{II} is the number of C₁-C₆ alkoxy groups per organic C₁-C₆ alkoxysilane (a1) used in agent (A), and
X is a number from about 0.1 to about 100.0, preferably from about 1.0 to about 50.0, more preferably from about 1.2 to about 30.0, still more preferably from about 1.3 to about 10, and most preferably from about 1.8 to about 4.5.
   Use of Catalysts (a3) for the Targeted Hydrolysis of the Organic C₁-C₆ Alkoxysilanes (a1) in the Agent (A).

In the course of the work leading to the present disclosure, it was found that the aminoalkyl-C₁-C₆-alkoxysilanes (a1) used in the agent (A) are so reactive that selective hydrolysis or pre-condensation with water (a2) proceeds at a sufficiently high rate even without the aid or presence of a catalyst.

Therefore, it is preferred to carry out the reaction of the C₁-C₆ organic alkoxysilane(s) (a1) with water (a2) without a catalyst (a3).

In principle, however, the use of a catalyst (a3) in the agent (A) is not excluded. By a catalyst (a3), the skilled person understands a substance that increases the reaction rate by lowering the activation energy of a chemical reaction without itself being consumed. The catalyst (a3) can be added before or after the water (a2) is added.

For the hydrolysis or pre-condensation of the mixtures of organic C₁-C₆ alkoxy siloxanes (a1), it has proved particularly advantageous to use a catalyst which can be dissolved or dispersed in water and is then added together with the water as a solution or dispersion to the mixture of organic C₁-C₆ alkoxy silanes and solvent.

Very preferably, the catalyst (a3) is selected from the group of inorganic or organic acids and inorganic or organic bases.

Particularly well-suited carotenes are inorganic and organic acids, which can preferably be selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid, and 1-hydroxyethane-1,1-diphosphonic acid. Explicitly, sulfuric acid, hydrochloric acid and maleic acid are particularly preferred.

Other particularly suitable catalysts are inorganic and organic bases, which can preferably be selected from the group of sodium hydroxide, potassium hydroxide, magnesium hydroxide and calcium hydroxide. Sodium hydroxide and potassium hydroxide are particularly preferred.

Other bases that can be used include ammonia, alkanolamines and/or basic amino acids.

Alkanolamines may be selected from primary amines having a C₂-C₆ alkyl parent bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

For the purposes of the present disclosure, an amino acid is an organic compound comprising in its structure at least one proton table amino group and at least one —COOH or one —SO₃H group. Preferred amino acids are aminocarboxylic acids, especially α-(alpha)-aminocarboxylic acids and ω-aminocarboxylic acids, whereby α-aminocarboxylic acids are particularly preferred.

As contemplated herein, basic amino acids are those amino acids which have an isoelectric point pI of greater than 7.0.

Basic α-aminocarboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, an agent as contemplated herein is therefore wherein the alkalizing agent is a basic amino acid from the group arginine, lysine, ornithine and/or histidine.

In addition, other inorganic alkalizing agents or bases can also be used. Inorganic alkalizing agents that can be used as contemplated herein can be selected, for example, from the group formed by sodium phosphate, potassium phosphate, sodium silicate, sodium metasilicate, potassium silicate, sodium carbonate and potassium carbonate.

In another very particularly preferred embodiment, a process as contemplated herein is wherein the catalyst is selected from the group of inorganic and organic bases, preferably from the group of sodium hydroxide, potassium hydroxide, magnesium hydroxide and calcium hydroxide.

In a further embodiment, a method as contemplated herein is wherein the first agent (A) is obtained by reacting one or more organic C₁-C₆ alkoxysilanes (a1) with water (a2) and one or more catalysts (a3), where:
the organic C₁-C₆ alkoxysilane or alkoxysilanes (a1) are selected from the group of (3-aminopropyl)triethoxysilane, (3-aminopropyl)trimethoxysilane, (2-aminoethyl)triethoxysilane, (2-aminoethyl)trimethoxysilane, (3-dimethylaminopropyl)triethoxysilane, (3-dimethylamino-propyl)tri-methoxysilane, (2-dimethylaminoethyl)triethoxysilane and (2-dimethylaminoethyl)trimethoxysilane; and
the catalyst or catalysts selected (a3) are selected from the group of inorganic acids, organic acids, inorganic bases and organic bases, preferably selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid and 1-hydroxyethane-1,1-diphosphonic acid, sodium hydroxide, potassium hydroxide, magnesium hydroxide and calcium hydroxide.

As contemplated herein, the catalysts (a3) are preferably used in the usual quantity ranges for catalysts. Since the catalysts (a3) accelerate the hydrolysis or condensation without being consumed themselves, the quantities used can be chosen to be correspondingly low.

Thus, the catalyst or catalysts (a3) can be used in an amount range from 0.0000001 to 2.0 wt. %, preferably from about 0.0001 to about 1.5 wt. % and very preferably from about 0.01 to about 1.0 wt. % in the agent (A). In this case, the figure in wt. % refers to the total amount of catalysts used in relation to the total amount of agent (A).

Absence of Organic C₁-C₆ Alkoxysilanes from Group (b1) in the Agent (A)

Essential to the process as contemplated herein is the separate preparation of the two silane blends (A) and (B), each of the two agents comprising C₁-C₆ alkoxysilanes of a particular group.

The first agent (A) comprises only the silanes (a1) of the aminoalkyl-C₁-C₆-alkoxysilane type, which are hydrolyzed and pre-condensed together. The second agent (B), on the other hand, comprises only silanes (b1) of the alkyl-C₁-C₆-alkoxysilane type. Both silane types (a1) and (b1) exhibit different reactivities, so that separating the two silane types from each other allows improved control of the two hydrolysis reactions.

If the two agents (A) and (B) were then mixed together after the separate hydrolysis and the cosmetic composition obtained with this mixture was later applied to the keratin material, it was observed that the results obtained were much more reproducible. For example, when the mixture of agents (A) and (B) was used in a hair dyeing process, both the intensity of the dyeing and its fastness properties were much more reproducible. In this way, the use of the mixture of (A) and (B) prepared by the process as contemplated herein ensures the production of hair dyeing with always the same color result, regardless of the climatic conditions prevailing during the application (such as humidity and temperature), the skill or speed of the user and the storage times of the agents.

In order to be able to carry out a separate hydrolysis of the two silane types (a1) and (b1) from each other, it is essential that the agent (A) does not contain any organic C₁-C₆ alkoxysilanes from the group (b1).

In other words, the method as contemplated herein is wherein the agent (A) is not a C₁-C₆ organic alkoxysilane (b1) selected from the group of methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, vinyltrimethoxysilane, vinyltriethoxysilane tetramethoxysilane and tetraethoxysilane.

In other words, the method as contemplated herein is wherein the total amount of C₁-C₆ organic alkoxysilane (b1) included in the agent (A) selected from the group of methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, vinyltrimethoxysilane, vinyltriethoxysilane, tetramethoxysilane and tetraethoxysilane—based on the total weight of the agent (A)—below about 0.01 wt. %., preferably below about 0.001 wt. % and very preferably at 0 wt. % (i.e., where agent (A) is substantially free from such compounds).

### Other Ingredients in the Agent (A)

In addition to the organic C₁-C₆ alkoxysilane(s) (a1) and water (a2), the agent (A) may optionally contain one or more further ingredients. For example, hydrolysis of C₁-C₆ alkoxysilanes (a1) may occur in the presence of one or more solvents.

Mixing can be accomplished, for example, by first placing the solvent other than water in a suitable reactor or reaction vessel and then adding the organic C₁-C₆ alkoxysilane(s). The addition can be done by dripping or pouring. Furthermore, it is also possible and as contemplated herein if at least one organic C₁-C₆ alkoxysilane is first introduced into the reaction vessel and then the solvent is added or added dropwise.

A sequential approach is also possible, i.e. first the addition of solvent and a first organic C₁-C₆ alkoxysilane, then again the addition of a solvent and then again the addition of another organic C₁-C₆ alkoxysilane.

The solvent is preferably added with stirring.

It may be preferred to select a solvent that has a boiling point at normal pressure (1013 hPa) of from about 20 to about 90° C., preferably from about 30 to about 85° C., and most preferably from about 40 to about 80° C.

Suitable solvents include:
Dichloromethane with a boiling point of 40° C. (1013 mbar)
Methanol with a boiling point of 65° C. (1013 mbar)
Tetrahydrofuran with a boiling point of 65.8° C. (1013 mbar)
Ethanol with a boiling point of 78° C. (1013 mbar)
Isopropanol with a boiling point of 82° C. (1013 mbar)
Acetonitrile with a boiling point of 82° C. (1013 mbar)

Furthermore, very particularly preferred solvents can be selected from the group of monohydric or polyhydric C₁-C₁₂ alcohols. Monohydric or polyhydric C₁-C₁₂ alcohols are compounds comprising one to twelve carbon atoms and bearing one or more hydroxyl groups. Other functional groups different from the hydroxy groups are not present in the C₁-C₁₂ alcohols as contemplated herein. The C₁-C₁₂ alcohols can be aliphatic or aromatic.

Suitable C₁-C₁₂ alcohols may include methanol, ethanol, n-propanol, isopropanol, n-pentanol, n-hexanol, benzyl alcohol, 2-phenylethanol, 1,2-propanediol, 1,3-propanediol and glycerol. Particularly suitable C₁-C₁₂ alcohols are methanol, ethanol and isopropanol.

However, in order to be able to control the hydrolysis of the C1-C6 alkoxysilanes (a1) by the water (a2) as precisely as possible, it has proved particularly advantageous either to add to the agent (A) only those further constituents which cannot react with the silanes (a1) in an unpredictable manner, or to add these further optional constituents to the agent (A) only in small to very small amounts. For this reason, it is particularly preferred if the agent (A) includes—based on its total weight—of at least 60% by weight, preferably at least 70 wt. %, more preferably at least 80% by weight, still more preferably at least 90% by weight and very particularly preferably at least 98% by weight of the silanes of group (a1) and water (a2).

In the context of a further very particularly preferred embodiment, a process as contemplated herein is wherein the total amount of the constituents (a1) and (a2) used in the agent (A) accounts for a proportion by weight of at least about 60 wt. %, preferably of at least about 70 wt. %, further preferably of at least about 80 wt. %, still further preferably of at least about 90 wt. % and very particularly preferably of at least about 98 wt. %, based on the total weight of the agent (A).

### Agent (B)

That the second agent (B) is obtained by reacting one or more organic C₁-C₆ alkoxysilanes (b1) with water (b2).

Organic C₁-C₆ Alkoxy Silanes (b2) in the Agent (B)

The organic C₁-C₆ alkoxy silane or silanes (b1) are organic, non-polymeric silicon compounds selected from alkyl-C₁-C₆ alkoxysilanes. Specifically, alkyl-C₁-C₆ alkoxysilanes utilizes as component (b1) are typically selected from the group of methyltrimethoxysilane, methyltriethoxy silane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, vinyltrimethoxysilane, vinyltriethoxysilane, tetramethoxysilane, and tetraethoxysilane.

The organic C₁-C₆ alkoxy silanes (b1) which are particularly suitable for solving the problem as contemplated herein are
Methyltrimethoxysilane:

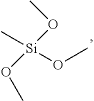

Methyltriethoxysilane:

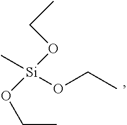

Ethyltrimethoxysilane:

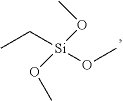

Ethyltriethoxysilane:

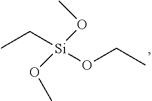

n-Hexyltrimethoxysilane (also known as hexyltrimethoxysilane):

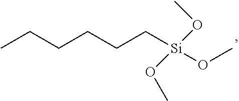

n-Hexyltriethoxysilane (also known as hexyltriethoxysilane):

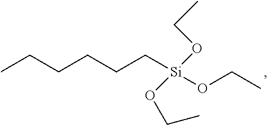 [SMILES: CCCCCC[Si](OCC)(OCC)OCC]

n-Octyltrimethoxysilane (also known as octyltrimethoxysilane):

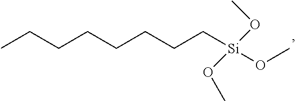

n-Octyltriethoxysilane (also known as octyltriethoxysilane):

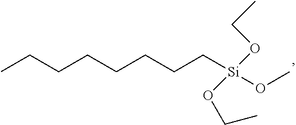 [SMILES: CCCCCCCC[Si](OC)(OCC)OCC]

n-Dodecyltrimethoxysilane (also known as dodecyltrimethoxysilane):

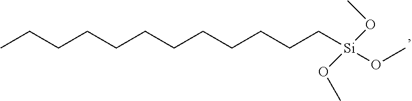 [SMILES: CCCCCCCCCCCC[Si](OC)(OC)OC]

n-Dodecyltriethoxysilane (also known as dodecyltriethoxysilane):

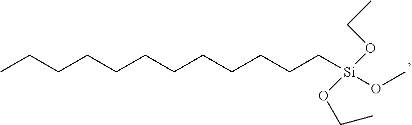 [SMILES: CCCCCCCCCCCC[Si](OC)(OCC)OCC]

Vinyl trimethoxysilane:

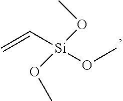

Vinyl triethoxysilane:

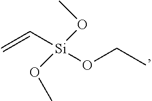

Tetramethoxysilane:

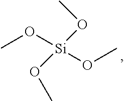

and
Tetraethoxysilane:

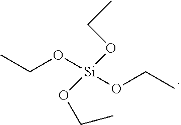

Also in the preparation of the agent (B), either only an organic C₁-C₆ alkoxysilane from the group (b1) or also the mixtures of the group can be used.

The silanes of group (b1) can be purchased commercially. For example, methyltriethoxysilane and methyltrimethoxysilane are available for purchase from chemical suppliers such as Acros, Sigma-Alrich, Fluka and VWR.

In the preparation of the first agent (B), one or more organic C₁-C₆ alkoxysilanes (b1) are reacted with water (b2). Particularly good results were obtained when methyltriethoxysilane and/or methyltri-methoxysilane were used as organic C₁-C₆ alkoxysilanes (b1). Most preferably, therefore, the second agent (B) is obtained by reacting methyltriethoxysilane and/or methyltrimethoxysilane (b1) with water (b2).

Selective Hydrolysis of the Organic C₁-C₆ Alkoxysilanes (b1) in the Agent (B) by Addition of Water (b2)

In the preparation of the agent (B), one or a mixture of several organic C₁-C₆ alkoxysilanes (b1) is mixed with water (b2) to initiate selective hydrolysis and, as a result, pre-condensation.

As previously described, hydrolysis of the alkoxysilanes (b1) in the agent (B) occurs separately from the silanes of group (a1) in the agent (A). Since only alkyl-C₁-C₆-alkoxy-silanes oligomerize with each other in agent (B), an oligomer is formed in agent (B) that is formed exclusively by selective hydrolysis and condensation of the alkyl-C₁-C₆-alkoxy-silanes. Thus, all monomer components of the oligomer or reaction product present in the agent (B) do not comprise aminoalkyl groups, but only alkyl groups.

The reaction with the water (b2) can be initiated, for example, by dropping or adding the water to the organic C₁-C₆ alkoxysilane(s) (b1). Optionally, a solvent may be present during the reaction or hydrolysis.

Also as contemplated herein, hydrolysis is started by adding the amount of water (b2) and the alkyl-C₁-C₆-alkoxy-silanes (b1).

Mixing with water (b2) can be done at room temperature. For the application properties of the subsequent cosmetic agent, it can be of further advantage if the mixture of organic C₁-C₆ alkoxysilanes (b1) and optionally solvent is heated to a temperature of from about 30 to about 80° C., preferably from about 40 to about 75° C., more preferably from about 45 to about 70° C. and very particularly preferably from about 50 to about 65° C., before the water (b2) is added.

Adjustment of the preferred and particularly preferred temperature ranges can be accomplished by tempering the reaction vessel or reactor. For example, the reaction vessel or reactor may be surrounded from the outside by a temperature control bath, which may be a water bath or silicone oil bath, for example.

If the reaction is carried out in a double-walled reactor, a temperature-controlled liquid can also be passed through the space formed by the two walls surrounding the reaction chamber.

Since the hydrolysis reaction is exothermic, it has been found to be particularly advantageous to stir or mix the reaction mixture for improved heat dissipation. It is therefore particularly preferred that the water be added while stirring. The reaction, which is now initiated by the addition of water and, if necessary, a catalyst, continues to proceed exothermically, so that the reaction mixture remains at the preferred temperature ranges indicated above or may even heat up further without any further energy being added. It is preferred if the additional heating due to the exothermic nature of the reaction remains within a range of from about 5 to about 20° C. If the reaction mixture heats up beyond this range, it is advantageous to cool the mixture.

The water can be added continuously, in partial quantities or directly as a total quantity. To ensure adequate temperature control, the amount and rate of water added is preferentially adjusted. Depending on the amount of silanes used, the addition and reaction can take place over a period of from about 2 minutes to about 72 hours.

The addition of the water (b2) initiates a selective hydrolysis of the organic C₁-C₆ alkoxysilanes (b1). For the purposes of the present disclosure, targeted hydrolysis means hydrolyzing some, but not all, of the C₁-C₆ alkoxy groups present in the C₁-C₆ organic alkoxysilanes.

Also in the preparation of agent (B), the alkoxysilane-water ratio has an influence on the crosslinking within the siloxane network. The degree of crosslinking can be described by so-called T-structures. Here, it T₀ means an unreacted monomer, e.g. methyltriethoxysilane or methyltrimethoxysilane. In the case of a T₁-bond, a siloxane bond exists between two alkoxysilanes, and the two siloxanes are not bonded to any other alkoxysilane. In an T₂-bond, one alkoxysilane is bonded to exactly two others. A T₃-bond describes a siloxane that is bonded to three other siloxanes.

By using different mole ratios of alkoxysilanes (b1) to water (b2), the distribution of these different T structures can be influenced. A high ratio, i.e. less water, leads to less cross-linked structures, whereas a low ratio, i.e. a larger amount of water, leads to more cross-linking.

The quantitative determination of the T0 structures, T1 structures, T2 structures and T3 structures included in the agent (B) in each case can be carried out, for example, by employing quantitative ²⁹Si NMR spectroscopy.
Use of NMR sample tubes
Device: Agilent, 600 MHz
Standard: TMS (tetramethylsilane)
Relaxation accelerator: Chromium(III) acetylacetonate
   ²⁹Si-NMR spectra were recorded in chloroform from each of the compositions. Measurements were taken on the day of production and after a defined period of time. By using the relaxation accelerator, the integrals of the individual signals became comparable with each other. The sum over all integrals was set equal to 100 mol %. For the quantitative determination, the area of each individual signal was related to the total sum over all integrals.

For example, the spectra can be measured using the procedure described in Journal of Organometallic Chemistry 625 (2001), 208-216.

The amount of water used to prepare the agent (B) is preferably equal to the molar amount of water determined according to equation (G-2):

Y=[(m_{I}(alkoxysilanes (b1))×m_{II}(alkoxy groups)]/ m_{II}(H₂O(b2)) (G-2),

where
m(H₂O) is the molar amount of water (b2) used in the agent (B),
m_{I} is the molar amount of the organic C₁-C₆ alkoxysilanes (b1) used in the agent (B),
m_{II} is the number of C₁-C₆ alkoxy groups per C₁-C₆ organic alkoxysilane (b1) used in the agent (B), and
Y represents a number.

In other words, the index number Y indicates the molar ratio of the total number of moles of hydrolyzable C₁-C₆ alkoxy groups (b1) in the agent (B), which is related to the molar amount of water (b2) used in the agent (B).

m_{I} indicates the total molar amount of C₁-C₆ organic alkoxysilanes (b1) used in the agent (B). If only one C₁-C₆ alkoxysilane (b1) of a particular structure is used, the total molar amount n_{I} is equal to the molar amount of the C₁-C₆ alkoxysilane used.

However, if a mixture of C₁-C₆ alkoxysilanes (b1) is used to prepare the agent (B), the total molar amount is the sum of the individual molar amounts of each C₁-C₆ alkoxysilane used.

Furthermore, m_{II} indicates the number of C₁-C₆ alkoxy groups per C₁-C₆ organic alkoxysilane (b1) used in the agent (B). If only one C₁-C₆ alkoxysilane of a given structure is used, m_{II} corresponds to the number of C₁-C₆ alkoxy groups present in that molecule.

However, if a mixture of C₁-C₆ alkoxysilanes (b1) is used to prepare the agent (B), the number of C₁-C₆ alkoxy groups (b₁) of each C₁-C₆ alkoxysilane enters the equation.

If several C₁-C₆ alkoxysilanes (b1) are used to prepare the agent (B), the above equation expands to equation (G-2′) by forming the respective summands:

$$Y = \frac{\sum [ mI ( Alkoxysilan ( b 1 ) ) \times mII ( Alkoxygruppen ) ]}{m ( H 2 O ) ( b 2 )} . \qquad ( G - {2}^{′} )$$

### Sample Calculation

In the preparation of agent (B), 15.0 g of methyltriethoxysilane (C₇H18O3Si=178.3 g/mol) and 15.0 g of methyltrimethoxysilane (C₄H12O3Si=136.22 g/mol) were mixed together. Then, 5.0 g of water (18.015 g/mol) was added with stirring.
15.0 g methyltriethoxysilane (MTES)=0.0841 mol
Methyltriethoxysilane has 3 hydrolyzable alkoxy groups per molecule.
15.0 g methyltrimethoxysilane (MTMS)=0.110 mol
Methyltrimethoxysilane has 3 hydrolyzable alkoxy groups per molecule.
5.0 g water=0.277 mol

When two different C₁-C₆ alkoxysilanes (b1) are used, the value Y is calculated by applying formula (G-2′) under formation of corresponding sums:

$$Y = \frac{[ m ( MTES ) \times 3 ] + ( m ( MTMS ) \times 3 ]}{m ( H 2 O ) ( b 2 )} , \\ Y = \frac{[ ( 0.0841 ) \times 3 ] + [ ( 0.11 ) \times 3 ]}{0.277} = 2.1 .$$

If further or other mixtures of C₁-C₆ alkoxy silanes (b1) are used, the formula (G-2) or (G-2′) is adapted accordingly or extended by the corresponding summands.

The degree of crosslinking set during preparation of the agent (B) by adding the appropriate amount of water (b2) influences the application properties of the coating produced during subsequent application to the keratin material. A particularly stable, and resistant film could be produced when second agent (B) is obtained by reacting with an amount of water equal to the molar amount of water determined according to equation (G-2), where Y is a number from about 0.1 to about 100.0, preferably from about 1.0 to about 50.0, more preferably from about 1.2 to about 30.0, still more preferably from about 1.3 to about 10, and most preferably from about 1.8 to about 4.5.

In the context of a further particularly preferred embodiment, a process as contemplated herein is therefore wherein the organic C₁-C₆-alkoxysilanes (b1) are reacted in the agent (B) with an amount of water (b2) corresponding to the molar amount of water determined according to equation (G-2):

Y=[(m_{I}(alkoxysilanes(b1))×m_{II}(alkoxy groups)]/m(H₂O(b2)) (G-2),

where
m(H₂O) is the molar amount of water (b2) used in the agent (B),
m_{I} is the molar amount of the organic C₁-C₆ alkoxysilanes (b1) used in the agent (B),
m_{II} is the number of C₁-C₆ alkoxy groups per C₁-C₆ organic alkoxysilane (b1) used in the agent (B), and
Y is a number from about 0.1 to about 100.0, preferably from about 1.0 to about 50.0, more preferably from about 1.2 to about 30.0, still more preferably from about 1.3 to about 10, and most preferably from about 1.8 to about 4.5.
   Use of Catalysts (b3) for the Targeted Hydrolysis of the Organic C₁-C₆ Alkoxysilanes (b1) in the Agent (B).

As the work leading to the present disclosure has shown, the alkyl-C₁-C₆ alkoxy silanes (b1) in the agent (B) have somewhat lower reactivity than the aminoalkyl-C₁-C₆ alkoxy silanes (a1) in the agent (A). While the hydrolysis of the more reactive silanes (a1) in the agent (A) starts very quickly even without a catalyst (a3) and proceeds at a sufficiently high rate, the use of a catalyst (b3) in the agent (B) has been found to be preferable for starting the hydrolysis reaction sufficiently quickly.

In the context of a further particularly preferred embodiment, a process as contemplated herein is therefore wherein the second agent (B) is obtained by reacting one or more organic C₁-C₆ alkoxysilanes (b1) with water (b2) and at least one catalyst (b3).

The catalyst (b3) is also understood as a substance or agent that increases the reaction rate by lowering the activation energy of a chemical reaction without being consumed itself. The catalyst (b3) can be added before or after the water (b2) is added.

For the hydrolysis or pre-condensation of the mixtures of organic C₁-C₆ alkoxy siloxanes (b1), it has proved particularly advantageous to use a catalyst which can be dissolved or dispersed in water and is then added together with the water as a solution or dispersion to the mixture of organic C₁-C₆ alkoxy silanes and solvent.

Very preferably, the catalyst (b3) is selected from the group of inorganic or organic acids and inorganic or organic bases.

Particularly well-suited carotenes are inorganic and organic acids, which can preferably be selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid, and 1-hydroxyethane-1,1-diphosphonic acid. Explicitly, sulfuric acid, hydrochloric acid and maleic acid are particularly preferred.

Other particularly suitable catalysts are inorganic and organic bases, which can preferably be selected from the group of sodium hydroxide, potassium hydroxide, magnesium hydroxide and calcium hydroxide. Sodium hydroxide and potassium hydroxide are particularly preferred.

Other bases that can be used include ammonia, alkanolamines and/or basic amino acids.

Alkanolamines may be selected from primary amines having a C₂-C₆ alkyl parent bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

For the purposes of the present disclosure, an amino acid is an organic compound comprising in its structure at least one proton table amino group and at least one —COOH or one —SO₃H group. Preferred amino acids are aminocarboxylic acids, especially α-(alpha)-aminocarboxylic acids and ω-aminocarboxylic acids, whereby α-aminocarboxylic acids are particularly preferred.

As contemplated herein, basic amino acids are those amino acids which have an isoelectric point pI of greater than 7.0.

Basic α-aminocarboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, an agent as contemplated herein is therefore wherein the alkalizing agent is a basic amino acid from the group arginine, lysine, ornithine and/or histidine.

In addition, other inorganic alkalizing agents or bases can also be used. Inorganic alkalizing agents that can be used as contemplated herein can be selected, for example, from the group formed by sodium phosphate, potassium phosphate, sodium silicate, sodium metasilicate, potassium silicate, sodium carbonate and potassium carbonate.

In another very particularly preferred embodiment, a process as contemplated herein is wherein the catalyst is selected from the group of inorganic and organic bases, preferably from the group of sodium hydroxide, potassium hydroxide, magnesium hydroxide and calcium hydroxide.

In a further embodiment, a process as contemplated herein is wherein the second agent (B) is obtained by reacting one or more organic C₁-C₆ alkoxysilanes (b1) with water (b2) and at least one catalyst (b3), the catalyst (b3) being selected from the group of inorganic and organic acids, preferably from the group comprising sulfuric acid, hydrochloric acid, phosphoric acid, maleic acid, citric acid, tartaric acid, malic acid, lactic acid, acetic acid, methanesulfonic acid, benzoic acid, malonic acid, oxalic acid and 1-hydroxyethane-1,1-diphosphonic acid.

As contemplated herein, the catalysts (b3) are preferably used in the usual quantity ranges for catalysts. Since the catalysts (b3) accelerate the hydrolysis or condensation without being consumed themselves, the quantities used can be chosen to be correspondingly low.

Thus, the catalyst or catalysts (b3) can be used in an amount range of from about 0.0000001 to about 2.0 wt. %, preferably from about 0.0001 to about 1.5 wt. % and most preferably from about 0.01 to about 1.0 wt. % in the agent (B). In this case, the figure in wt. % refers to the total amount of catalysts used in relation to the total amount of agent (B).

Absence of organic C₁-C₆ alkoxysilanes from group (b1) in the agent (A)

Essential to the process as contemplated herein is the separate preparation of the two silane blends (A) and (B), each of the two agents comprising C₁-C₆ alkoxysilanes of a particular group.

Just as the first agent (A) comprises only silanes (a1) of the aminoalkyl-C₁-C₆-alkoxysilane type, which are hydrolyzed and pre-condensed with each other, the second agent (B) comprises only silanes (b1) of the alkyl-C₁-C₆-alkoxysilane type.

Accordingly, it is characteristic of the process as contemplated herein that the agent (B) is substantially free from, alternatively does not contain any, organic C₁-C₆ alkoxysilanes from the group (a1).

In other words, the process as contemplated herein is wherein the agent (B) is not a C₁-C₆ organic alkoxysilane (a1) selected from the group of (3-aminopropyl)triethoxysilane, (3-aminopropyl)trimethoxysilane, (2-aminoethyl)triethoxysilane, (2-aminoethyl)trimethoxysilane, (3-dimethylaminopropyl)triethoxysilane, (3-dimethylamino-propyl)tri-methoxysilane, (2-dimethylaminoethyl)trimethoxysilane and (2-dimethylaminoethyl)trimethoxysilane.

In still other words, the process as contemplated herein is wherein the total amount of organic C₁-C₆ alkoxysilanes (a1) included in the agent (B) selected from the group of (3-aminopropyl)triethoxysilane, (3-aminopropyl)trimethoxysilane, (2-aminoethyl)triethoxysilane, (2-aminoethyl)trimethoxysilane, (3-dimethylaminopropyl)triethoxysilane, (3-dimethylamino-propyl)tri-methoxysilane, (2-dimethylaminoethyl)trimethoxysilane and (2-dimethylaminoethyl)trimethoxysilane—based on the total weight of the agent (B)—is below about 0.01% by weight.—%, preferably below about 0.001% by weight and very preferably at 0% by weight (i.e., agent (B) is substantially free from such compounds).

### Other Ingredients in the Agent (B)

In addition to the organic C₁-C₆ alkoxysilane(s) (b1) and water (b2), agent (B) may optionally also contain one or more further ingredients, as in agent (A). For example, hydrolysis of C₁-C₆ alkoxysilanes (b1) may occur in the presence of one or more solvents.

Mixing can be accomplished, for example, by first placing the solvent other than water in a suitable reactor or reaction vessel and then adding the organic C₁-C₆ alkoxysilane(s). The addition can be done by dripping or pouring. Furthermore, it is also possible and as contemplated herein if at least one organic C₁-C₆ alkoxysilane is first introduced into the reaction vessel and then the solvent is added or added dropwise.

A sequential approach is also possible, i.e. first the addition of solvent and a first organic C₁-C₆ alkoxysilane, then again the addition of a solvent and then again the addition of another organic C₁-C₆ alkoxysilane.

The solvent is preferably added with stirring.

It may be preferred to select a solvent that has a boiling point at normal pressure (1013 hPa) of from about 20 to about 90° C., preferably from about 30 to about 85° C., and most preferably from about 40 to about 80° C.

Suitable solvents include:
Dichloromethane with a boiling point of 40° C. (1013 mbar)
Methanol with a boiling point of 65° C. (1013 mbar)
Tetrahydrofuran with a boiling point of 65.8° C. (1013 mbar)
Ethanol with a boiling point of 78° C. (1013 mbar)
Isopropanol with a boiling point of 82° C. (1013 mbar)
Acetonitrile with a boiling point of 82° C. (1013 mbar)

Furthermore, very particularly preferred solvents can be selected from the group of monohydric or polyhydric C₁-C₁₂ alcohols. Monohydric or polyhydric C₁-C₁₂ alcohols are compounds comprising one to twelve carbon atoms and bearing one or more hydroxyl groups. Other functional groups different from the hydroxy groups are not present in the C₁-C₁₂ alcohols as contemplated herein. The C₁-C₁₂ alcohols can be aliphatic or aromatic.

Suitable C₁-C₁₂ alcohols may include methanol, ethanol, n-propanol, isopropanol, n-pentanol, n-hexanol, benzyl alcohol, 2-phenylethanol, 1,2-propanediol, 1,3-propanediol and glycerol. Particularly suitable C₁-C₁₂ alcohols are methanol, ethanol and isopropanol.

However, in order to be able to control the hydrolysis of the C₁-C₆ alkoxysilanes (b1) by the water (b2) as precisely as possible, it has proved particularly advantageous either to add to the agent (B) only those further constituents which cannot react with the silanes (b1) in an unpredictable manner, or to add further constituents to the agent (B) only in small to very small amounts. For this reason, it is particularly preferred if the agent (B) of at least about 60 wt. %, preferably at least about 70 wt. %, more preferably at least about 80 wt. %, still more preferably at least about 90 wt. % and very particularly preferably at least about 98 wt. % of the silanes of group (b1) and water, based on its total weight.

In the context of a further very particularly preferred embodiment, a process as contemplated herein is wherein the total amount of the constituents (b1) and (b2) used in the agent (B) constitutes a proportion by weight of at least about 60 wt. %, preferably of at least about 70 wt. %, further preferably of at least about 80 wt. %, still further preferably of at least about 90 wt. % and very particularly preferably of at least about 98 wt. %, based on the total weight of the agent (B).

Reaction of the Organic C₁-C₆ Alkoxy Silanes with Water in Agents (A) and (B).

In agents (A) and (B), the reaction of the organic C₁-C₆ alkoxy silanes with water can take place in different ways. The reaction starts as soon as the C₁-C₆ alkoxy silanes come into contact with water by mixing. As soon as C₁-C₆ alkoxy silanes and water come into contact, an exothermic hydrolysis reaction takes place according to the following scheme

Agent (A): Reaction scheme using the example of 3-aminopropyltriethoxysilane:

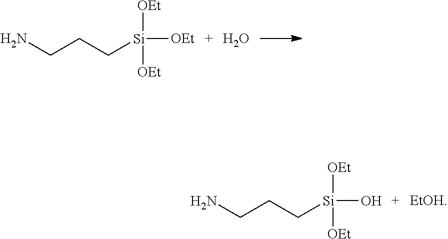 [SMILES: CCO[Si](CCCN)(OCC)OCC ; O ; CCO[Si](O)(CCCN)OCC ; "EtOH."]

Depending on the number of hydrolyzable C₁-C₆ alkoxy groups per silane molecule, the hydrolysis reaction can also occur several times per C₁-C₆ alkoxy silane used:

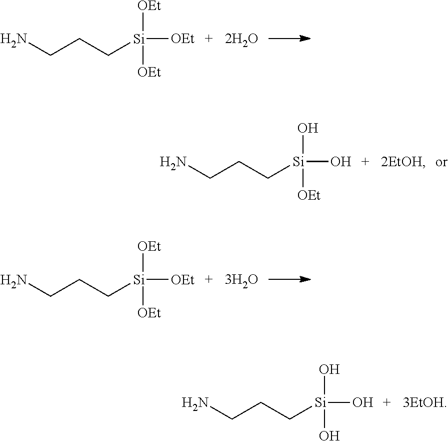

Agent (B): Hydrolysis using the example of methyltrimethoxysilane:

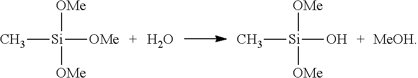

Depending on the amount of water used, the hydrolysis reaction can also take place several times per C₁-C₆ alkoxy silane used:

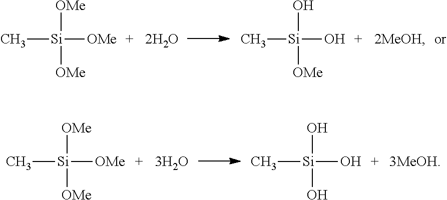

Following the hydrolysis or quasi simultaneously with the hydrolysis, condensation of the partially (or in parts completely) hydrolyzed C₁-C₆ alkoxy silanes takes place. The pre-condensation can proceed, for example, according to the following scheme:

Agent (A): Condensation using the example of 3-aminopropyltriethoxysilane:

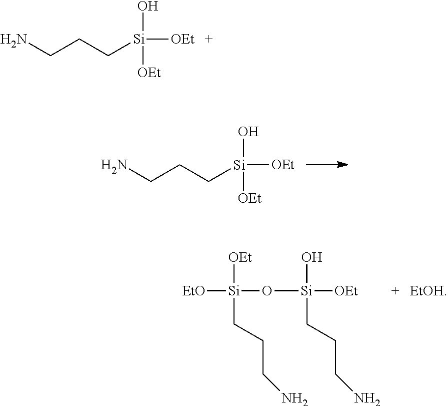 [SMILES: CCO[Si](O)(CCCN)OCC ; CCO[Si](O)(CCCN)OCC ; CCO[Si](O)(CCCN)O[Si](CCCN)(OCC)OCC ; "EtOH."]

Both partially hydrolyzed and fully hydrolyzed C₁-C₆ alkoxysilanes can participate in the condensation reaction, undergoing condensation with not yet reacted, partially or also fully hydrolyzed C₁-C₆ alkoxysilanes. Possible condensation reactions are for example:

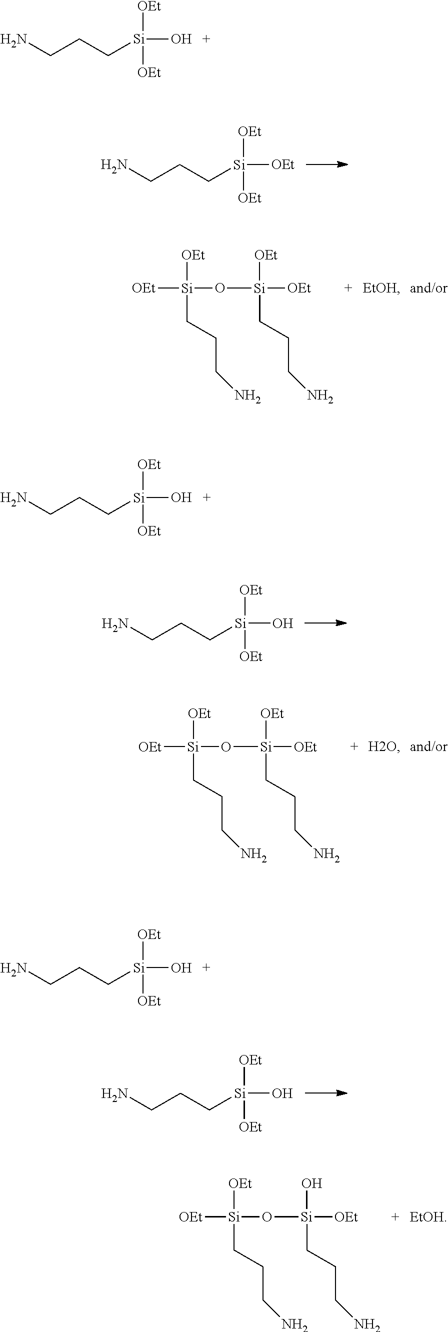

Agent (B): Condensation using the example of methyltrimethoxysilane

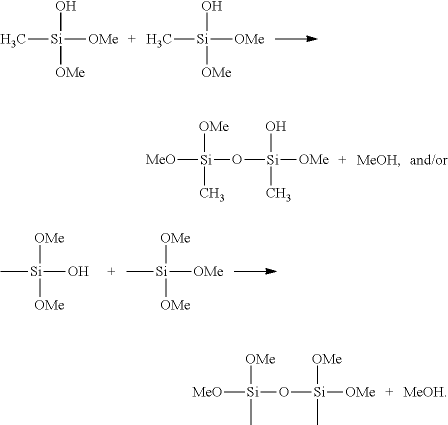

In the above exemplary reaction schemes, the condensation to a dimer is shown in each case, but further condensations to oligomers with several silane atoms are also possible and also preferred, since they lead to the crosslinking of the siloxane mixture described above.

### Reaction Vessels

Agents (A) and (B) are preferably prepared from organic C₁-C₆ alkoxysiloxanes in a reactor or reaction vessel suitable for this purpose. A reaction vessel that is very suitable for smaller preparations is, for example, a glass flask commonly used for chemical reactions with a capacity of 1 liter, 3 liters or 5 liters, such as a 3-liter single-neck or multi-neck flask with ground joints.

A reactor is a confined space (container, vessel) that has been specially designed and manufactured to allow certain reactions to take place and be controlled under defined conditions.

For larger approaches, it has proven advantageous to carry out the reaction in reactors made of metal. Typical reactors may include, for example, a 10-liter, 20-liter, or 50-liter capacity. Larger reactors for the production area can also include fill volumes of about 100-liters, about 500-liters, or about 1000-liters.

Double-wall reactors have two reactor shells or reactor walls, with a tempering fluid circulating in the area between the two walls. This enables particularly good adjustment of the temperature to the required values.

The use of reactors, in particular double-walled reactors with an enlarged heat exchange surface, has also proven to be particularly suitable, whereby the heat exchange can take place either through internal installations or through the use of an external heat exchanger.

Corresponding reactors are, for example, laboratory reactors from the company IKA. In this context, the models “LR-2.ST” or the model “magic plant” can be mentioned.

Other reactors that can be used are reactors with thin-film evaporators, since this allows very good heat dissipation and thus particularly precise temperature control. Thin film evaporators are alternatively referred to as thin film evaporators. Thin film evaporators can be purchased commercially from Asahi Glassplant Inc. for example.

### Mixing the Agents (A) and (B)

The cosmetic composition as contemplated herein is now obtained by mixing a first agent (A) with a second agent (B).

As contemplated herein, this mixing takes place before the cosmetic composition is applied to the keratin material. Here, the mixing of agents (A) and (B) can either take place shortly before use, or the two agents (A) and (B) can, for example, first be produced separately, then stored for a certain period of time, and then mixed together. After this mixing process, the cosmetic preparation can be stored again for a certain period of time before it is then applied to the keratin material. As previously described, a repeat hydrolysis reaction can also be carried out with mixture (A)+(B).

Agents (A) and (B) may first be stored for a few days, weeks or months, for example, before being mixed together. Also, the cosmetic preparation prepared by mixing agents (A) and (B) can be stored again for some time, for example, a days, weeks or months.

The degree of crosslinking in the cosmetic composition as contemplated herein also depends on the mixing ratio in which the two agents (A) and (B) are mixed together. In order to produce particularly stable, resistant and reproducible films or coatings on the keratin material, it has proved particularly preferable to mix the agents (A) and (B) in a specific ratio.

Particularly good results were obtained when the agent (A) and the agent (B) were mixed together in a weight ratio (A)/(B) of from about 1:5 to about 5:1, preferably from about 1:4 to about 4:1, more preferably from about 1:3 to about 3:1, and most preferably from about 1:2 to about 2:1.

In a particularly preferred embodiment, a cosmetic composition as contemplated herein is wherein it is obtained by mixing the first agent (A) with the second agent (B) in a weight ratio (A)/(B) of from about 1:5 to about 5:1, preferably from about 1:4 to about 4:1, more preferably from about 1:3 to about 3:1, and most preferably from about 1:2 to about 2:1.

### Sample Calculation

First, the two agents (A) and (B) were prepared as described previously, filled into separate containers, and stored for several days. Then 50 g of agent (A) was mixed with 50 g of agent (B). The weight ratio (A)/B) is 50/50 and is therefore 1:1. This mixture of (A) and (B) can either be bottled and stored for use as-is, or the mixture is subjected to another hydrolysis reaction by adding a further amount of water.

### Process for the Preparation of the Cosmetic Composition

The process for preparing the cosmetic composition as contemplated herein may comprise various steps.

Particularly preferred is A as contemplated herein comprising the following steps.
(1) Provide one or more organic C₁-C₆ alkoxysilanes (a1) in a reaction vessel,
(2) Preparation of the agent (A) by mixing the organic C₁-C₆ alkoxy silanes (a1) with water (a2), optionally with stirring and/or heating the mixture in the reaction vessel to a temperature of from about 30 to about 90° C.,
(3) Providing one or more organic C₁-C₆ alkoxysilanes (b1) in a reaction vessel different from the reaction vessel in step (1),
(4) Preparation of the agent (B) by mixing the organic C₁-C₆ alkoxy silanes (b1) with water (b2), optionally with stirring and/or heating the mixture in the reaction vessel to a temperature of from about 30 to about 90° C.,
(5) Mixing of agents (A) and (B), optionally with stirring and/or heating of the mixture in the reaction vessel to a temperature of from about 30 to about 90° C.

Following the mixing of agents (A) and (B) in step (5), the mixture thus obtained can either be bottled for storage or for making the product available, or it can be followed by a further reaction in which at least one substance selected from the group of water, alkalizing agent(s), acid(s) and solvent(s) is added.

Accordingly, a very particularly preferred method is one which further comprises:
(6) adding water and/or at least one alkalizing agent and/or at least one acid and/or at least one solvent to the mixture of agents (A) and (B) and/or the
(7) bottling (i.e., discharging and storing) the mixture obtained in step (5) or in step (6) from the reaction vessel.
   Particularly well-suited alkalizing agents, which can alternatively be referred to as bases, are those described earlier. Particularly well suited acids and/or solvents are those described earlier.

In the context of a further very particularly preferred embodiment, a process as contemplated herein is wherein the alkalizing agent is selected from the group of sodium hydroxide, potassium hydroxide, lithium hydroxide, magnesium hydroxide and calcium hydroxide.

In another particularly preferred embodiment, a process as contemplated herein is wherein the solvent is selected from the group of poly-C₁-C₆-alkylene glycols, 1,2-propylene glycol, 1,3-propylene glycol, 1,2-butylene glycol, dipropylene glycol, ethanol, isopropanol, diethylene glycol monoethyl ether, glycerol, phenoxyethanol and benzyl alcohol.

Furthermore, a method comprising the following steps is also particularly suitable:
(1) Provide one or more organic C₁-C₆ alkoxysilanes (a1) in a reaction vessel,
(2) Preparation of the agent (A) by mixing the organic C₁-C₆ alkoxy silanes (a1) with water (a2), optionally with stirring and/or heating the mixture in the reaction vessel to a temperature of from about 30 to about 90° C.,
(3) Providing one or more organic C₁-C₆ alkoxysilanes (b1) in a reaction vessel different from the reaction vessel in step (1),
(4) Mixing one or more catalysts (b3) with water (b2),
(5) Preparation of the agent (B) by mixing the organic C₁-C₆ alkoxy silanes (b1) with the mixture of catalyst (b3) and water (b2), optionally with stirring and/or heating the mixture in the reaction vessel to a temperature of from about 30 to about 90° C.,
(6) Mixing of agents (A) and (B), optionally with stirring and/or heating of the mixture in the reaction vessel to a temperature of from about 30 to about 90° C.

In these embodiments, the mixing of agents (A) and (B), which may be carried out with stirring and/or heating of the mixture in the reaction vessel to a temperature of from about 30 to about 90° C., may also be followed by a further reaction step.

Accordingly, a very particularly preferred method is one which further comprises:
(7) adding water and/or at least one alkalizing agent and/or at least one acid and/or at least one solvent to the mixture of agents (A) and (B) and/or the
(8) Filling the mixture obtained in step (6) or in step (7) from the reaction vessel.

Further suitable is also a method comprising the following steps:
(1) Provide one or more organic C₁-C₆ alkoxysilanes (a1) in a reaction vessel,
(2) Mixing one or more catalysts (a3) with water (a2),
(3) Preparation of the agent (A) by mixing the organic C₁-C₆ alkoxy silanes (a1) with the mixture of catalyst (a3) and water (a2), optionally with stirring and/or heating the mixture in the reaction vessel to a temperature of from about 30 to about 90° C.,
(4) Providing one or more organic C₁-C₆ alkoxysilanes (b1) in a reaction vessel different from the reaction vessel in step (1),
(5) Mixing one or more catalysts (b3) with water (b2),
(6) Preparation of the agent (B) by mixing the organic C₁-C₆ alkoxy silanes (b1) with the mixture of catalyst (b3) and water (b2), optionally with stirring and/or heating the mixture in the reaction vessel to a temperature of from about 30 to about 90° C.,
(7) Mixing of agents (A) and (B), optionally with stirring and/or heating of the mixture in the reaction vessel to a temperature of from about 30 to about 90° C.

In these embodiments, the mixing of agents (A) and (B), which may be carried out with stirring and/or heating of the mixture in the reaction vessel to a temperature of from about 30 to about 90° C., may also be followed by a further reaction step.

Accordingly, a very particularly preferred method is one which further comprises:
(8) Adding water and/or an alkalizing agent and/or an acid and/or at least one solvent to the mixture of agents (A) and (B) and/or the
(9) Bottling the mixture obtained in step (7) or in step (8) from the reaction vessel.

### Cosmetic Composition for the Treatment of Keratinous Material

The cosmetic compositions prepared via the process of the first subject present disclosure are particularly suitable for treating keratinous material.

A second object of the present disclosure is therefore a cosmetic composition for the treatment of keratinous material, in particular human hair, prepared according to a process as disclosed in detail in the description of the first subject matter of the present disclosure.

### Use of the Cosmetic Composition for the Treatment of Keratinous Material

The cosmetic composition prepared via the process of the first present disclosure is particularly suitable for treating keratinous material, in particular for coloring keratinous material, in particular for coloring human hair.

A third object of the present disclosure is therefore the use of a cosmetic composition prepared by a process as disclosed in detail in the description of the first object of the present disclosure for the treatment of keratinous material, in particular for coloring keratinous material, in particular for coloring human hair.

Concerning the further preferred embodiments of the cosmetic composition as contemplated herein and its use, mutatis mutandis what has been said about the method as contemplated herein applies.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A process for preparing a cosmetic composition for treating keratinous material, comprising:
preparing a first agent (A) by reacting one or more aminoalkyl-C₁-C₆ alkoxysilanes (a1) with water (a2),
wherein each of the one or more aminoalkyl-C₁-C₆ alkoxysilanes (a1) is independently selected from (3-aminopropyl)triethoxysilane, (3-aminopropyl)trimethoxysilane, (2-aminoethyl)triethoxysilane, (2-aminoethyl)trimethoxysilane, (3-dimethylaminopropyl)triethoxysilane, (3-dimethylamino-propyl)trimethoxysilane, (2-dimethylaminoethyl)triethoxysilane, and (2-dimethylaminoethyl)trimethoxysilane, and
wherein the first agent (A) is substantially free from alkyl-C₁-C₆ alkoxysilanes;
preparing a second agent (B) by reacting one or more alkyl-C₁-C₆ alkoxysilanes (b1) with water (b2),
wherein each of the one or more alkyl-C₁-C₆ alkoxysilanes (b1) is independently selected from a Group (b1): methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, vinyltrimethoxysilane, vinyltriethoxysilane tetramethoxysilane, and tetraethoxysilane, and
wherein the second agent (B) is substantially free from aminoalkyl-C₁-C₆ alkoxysilanes; and
mixing together the first agent (A) and the second agent (B), thereby preparing the cosmetic composition.

2. The process of claim 1, wherein preparing the first agent (A) comprises reacting the one or more aminoalkyl-C₁-C₆ alkoxysilanes (a1) with water (a2) in molar amounts according to equation (G-1):
X=[(n_{I}(alkoxysilanes(a1))×n_{II}(alkoxy groups)]/n(H₂O) (G-1),
where:
n(H₂O)is the molar amount of water (a2),
n_{I} is the molar amount of the one or more aminoalkyl-C₁-C₆ alkoxysilanes (a1),
n_{II} is the number of C₁-C₆ alkoxy groups per molecule aminoalkyl-C₁-C₆ alkoxysilanes in (a1), and
X is a number from 0.1 to 100.0.

3. The process of claim 1, wherein the total amount of the one or more aminoalkyl-C₁-C₆ alkoxysilanes (a1) and water (a2) used to prepare the agent (A) is a proportion by weight of at least about 60 wt. %, based on the total weight of the agent (A).

4. The process of claim 1, wherein preparing the second agent (B) comprises reacting the one or more alkyl-C₁-C₆ alkoxysilanes (b1) with water (b2) in molar amounts according to equation (G-2):
Y=[(m_{I}(alkoxysilanes (b1))×m_{II}(alkoxy groups)]/m(H₂O) (G-2),
where:
m(H₂O) is the molar amount of water (b2),
m_{I} is the molar amount of the one or more alkyl-C₁-C₆ alkoxysilanes (b1),
m_{II} is the number of C₁-C₆ alkoxy groups per molecule alkyl-C₁-C₆ alkoxysilanes in (b1), and
Y is a number from 0.1 to 100.0.

5. The process of claim 1, wherein preparing the second agent (B) comprises reacting the one or more alkyl-C₁-C₆ alkoxysilanes (b1) with water (b2) in the presence of at least one catalyst (b3).

6. The process of claim 5, wherein the catalyst (b3) is selected from inorganic and organic acids.

7. The process of claim 1, wherein the total amount of the one or more alkyl-C₁-C₆ alkoxysilanes (b1) and water (b2) used to prepare the second agent (B) is a proportion by weight of at least about 60 wt. %, based on the total weight of the second agent (B).

8. The process of claim 1, wherein:
preparing the first agent (A) comprises:
(1) providing the one or more aminoalkyl-C₁-C₆ alkoxysilanes (a1) in a first reaction vessel, and
(2) mixing the one or more aminoalkyl-C₁-C₆ alkoxysilanes (a1) with water (a2), optionally with stirring and/or heating the mixture in the first reaction vessel to a temperature of from about 30 to about 90° C., to give the first agent (A);
preparing the second agent (B) comprises:
(3) providing the one or more alkyl-C₁-C₆ alkoxysilanes (b1) in a second reaction vessel, and
(4) mixing the alkyl-C₁-C₆ alkoxysilanes (b1) with water (b2), optionally with stirring and/or heating the mixture in the second reaction vessel to a temperature of from about 30 to about 90° C., to give the second agent (B); and
mixing together the first agent (A) and the second agent (B) comprises:
(5) combining the first agent (A) and the second agent (B) in a reaction vessel to give a mixture, optionally with stirring and/or heating of the mixture in the reaction vessel to a temperature of from about 30 to about 90° C., thereby preparing the cosmetic composition.

9. The process of claim 8, further comprising:
(6) adding at least one of water, an alkalizing agent, an acid, and/or a solvent to the reaction vessel with the mixture of the first agent (A) and the second agent (B); and/or
(7) discharging a product obtained in (6) from the reaction vessel, thereby preparing the cosmetic composition.

10. The process of claim 9, wherein the alkalizing agent is added to the reaction vessel with the mixture of the first agent (A) and the second agent (B), and wherein the alkalizing agent is selected from sodium hydroxide, potassium hydroxide, lithium hydroxide, magnesium hydroxide, calcium hydroxide, and combinations thereof

11. The process of claim 9, wherein the solvent is added to the reaction vessel with the mixture of the first agent (A) and the second agent (B), and wherein, and wherein the solvent is selected from poly-C₁-C₆ alkylene glycols, 1,2-propylene glycol, 1,3-propylene glycol, 1,2-butylene glycol, dipropylene glycol, ethanol, isopropanol, diethylene glycol monoethyl ether, glycerol, phenoxyethanol, benzyl alcohol, and combinations thereof.

12. A cosmetic composition for treating keratinous material prepared by the process of claim 1.

13. A method of coloring keratinous material, comprising:
applying a cosmetic composition to the keratinous material, wherein the cosmetic composition prepared by the process of claim 1.

14. The process of claim 1, wherein:
the total amount of the one or more aminoalkyl-C₁-C₆ alkoxysilanes (a1) and water (a2) used to prepare the agent (A) is a proportion by weight of at least about 90 wt. %, based on the total weight of the agent (A);
the total amount of the one or more alkyl-C₁-C₆ alkoxysilanes (b1) and water (b2) used to prepare the second agent (B) is a proportion by weight of at least about 90 wt. %, based on the total weight of the second agent (B); and
the first agent (A) and the second agent (B) are mixed together in a weight ratio (w/w) of from about 1:5 to about 5:1 (A):(B).

15. The process of claim 2, wherein X is from about 1.2 to about 30.

16. The process of claim 3, wherein the total amount of the one or more aminoalkyl-C₁-C₆ alkoxysilanes (a1) and water (a2) used to prepare the agent (A) is a proportion by weight of at least about 80 wt. %, based on the total weight of the agent (A).

17. The process of claim 4, wherein Y is from about 1.2 to about 30.

18. The process of claim 7, wherein the total amount of the one or more alkyl-C₁-C₆ alkoxysilanes (b1) and water (b2) used to prepare the second agent (B) is a proportion by weight of at least about 80 wt. %, based on the total weight of the second agent (B).

19. The process of claim 8, wherein in (2) the mixture of the one or more aminoalkyl-C₁-C₆ alkoxysilanes (a1) and the water (a2) is substantially free from any catalyst (b3).

20. The process of claim 8, wherein mixing the one or more alkyl-C₁-C₆ alkoxysilanes (b1) with water (b2) in (4) further comprises combining the water (b2) with at least one catalyst (b3) and subsequently adding the combination to the one or more one or more organic C₁-C₆ alkoxy silanes (b1) in the second reaction vessel.
